(19) United States
(12) Patent Application Publication
(10) Pub. No.: US 2025/0288210 A1
(43) Pub. Date: Sep. 18, 2025

(54) BLOOD PRESSURE CALIBRATION

(71) Applicant: Oura Health Oy, Oulu (FI)

(72) Inventors: Antti Aleksi Rantanen, Oulu (FI); Iman Alikhani, Espoo (FI)

(21) Appl. No.: 18/608,228

(22) Filed: Mar. 18, 2024

(51) Int. Cl. A61B 5/0205; A61B 5/00; A61B 5/021; A61B 5/024; A61B 5/11

(52) U.S. Cl. CPC A61B 5/0205; A61B 5/11; A61B 5/681; A61B 5/742; A61B 5/02125; A61B 5/02416; A61B 5/02438; A61B 2560/0223; A61B 2562/0219

(57) ABSTRACT

Methods, systems, and devices for calibrating blood pressure measurements of a wearable device are described. The wearable device may acquire, during a calibration procedure, reference photoplethysmography (PPG) data from a body part of a user using a wearable device while the body part is at different positions (e.g., heights) relative to a heart of the user. The wearable device may determine a calibration function associated with blood pressure metrics determined based on the reference PPG data. The wearable device may then acquire additional PPG data from the body part of the user using the wearable device during an additional time interval and determine a position of the body part of the user during the additional time interval based on the reference PPG data. The wearable device may determine a blood pressure metric for the user based on the additional PPG data and the calibration function.

### FIELD OF TECHNOLOGY

The following relates to wearable devices and data processing, including techniques for blood pressure calibration.

### BACKGROUND

Some wearable devices may be configured to collect data from users, which may include photoplethysmography (PPG) data, temperature data, heart rate data, and the like. In some examples, the PPG data may be used to determine information regarding blood pressure associated with users.

### BRIEF DESCRIPTION OF THE DRAWINGS

FIG. 1 illustrates an example of a system that supports techniques for blood pressure calibration in accordance with aspects of the present disclosure.

FIG. 2 illustrates an example of a system that supports techniques for blood pressure calibration in accordance with aspects of the present disclosure.

FIG. 3 shows an example of a system that supports techniques for blood pressure calibration in accordance with aspects of the present disclosure.

FIG. 4 shows an example of PPG diagrams that support techniques for blood pressure calibration in accordance with aspects of the present disclosure.

FIG. 5 shows an example of a graphic user interface (GUI) that supports techniques for blood pressure calibration in accordance with aspects of the present disclosure.

FIG. 6 shows an example of a flowchart that supports techniques for blood pressure calibration in accordance with aspects of the present disclosure.

FIG. 7 shows a block diagram of an apparatus that supports techniques for blood pressure calibration in accordance with aspects of the present disclosure.

FIG. 8 shows a block diagram of a wearable device manager that supports techniques for blood pressure calibration in accordance with aspects of the present disclosure.

FIG. 9 shows a diagram of a system including a device that supports techniques for blood pressure calibration in accordance with aspects of the present disclosure.

FIG. 10 shows a flowchart illustrating methods that support techniques for blood pressure calibration in accordance with aspects of the present disclosure.

### DETAILED DESCRIPTION

Wearable devices may be configured to measure blood flow using photoplethysmography (PPG) to provide insight into blood pressure changes for a user. In some cases, however, local blood flow may be affected by a position of a part of a user's body in relation to the heart due to effects of gravity. For example, if a user's arm is raised above the heart, arterial pressure within the user's arm may decrease, while venous blood flow may increase. Conversely, if the user's arm is lowered below the heart, the arterial pressure may increase, and venous blood flow may decrease. As such, the “observed” arterial blood pressure within the user's arm may change, even though the user's “actual” arterial blood pressure at their heart remains constant. Accordingly, blood pressure measurements observed by wearable devices may be affected by a user's movements and relative positioning of the body part being used to perform blood pressure measurements, and techniques for adjusting and calibrating blood pressure measurements may be desired.

In accordance with examples as described herein, a wearable device may be calibrated to increase the accuracy of blood pressure measurements performed by the wearable device. In some examples, the calibration techniques described herein may be used to calibrate “observed” blood pressure measurements based on a relative position of the user's body part used to perform the blood pressure measurements. For example, a user may be instructed (e.g., via a GUI) to place their hand at heart level, above heart level (e.g., with the user's arm stretched upwards), and below heart level (e.g., with the user's arm straight down by the user's side) for some time duration. The wearable device may perform PPG measurements at each of these locations (e.g., above, below, and at heart level), and calculate blood pressure measurements for each of the respective positions. As such, by performing the calibration procedure and performing measurements with the user's arm at varying levels, the wearable system may “learn” how blood pressure measurements taken above/below the user's heart relate to the user's “actual” blood pressure, and the wearable device may therefore adjust or calibrate future blood pressure measurements to report a blood pressure metric more accurately. In particular, the wearable system may “learn” how the user's PPG morphology changes when the user's arm is at, above, and below heart level. As such, the PPG morphology may be used in the future to estimate the user's hand position, and modify/calibrate determined blood pressure metrics based on the hand position.

Aspects of the disclosure are initially described in the context of systems supporting physiological data collection from users via wearable devices. Aspects of the disclosure are additionally described with respect to PPG diagrams and GUIs that illustrate aspects of the blood pressure calibration techniques described herein. Aspects of the disclosure are further illustrated by and described with reference to apparatus diagrams, system diagrams, and flowcharts that relate to blood pressure calibration.

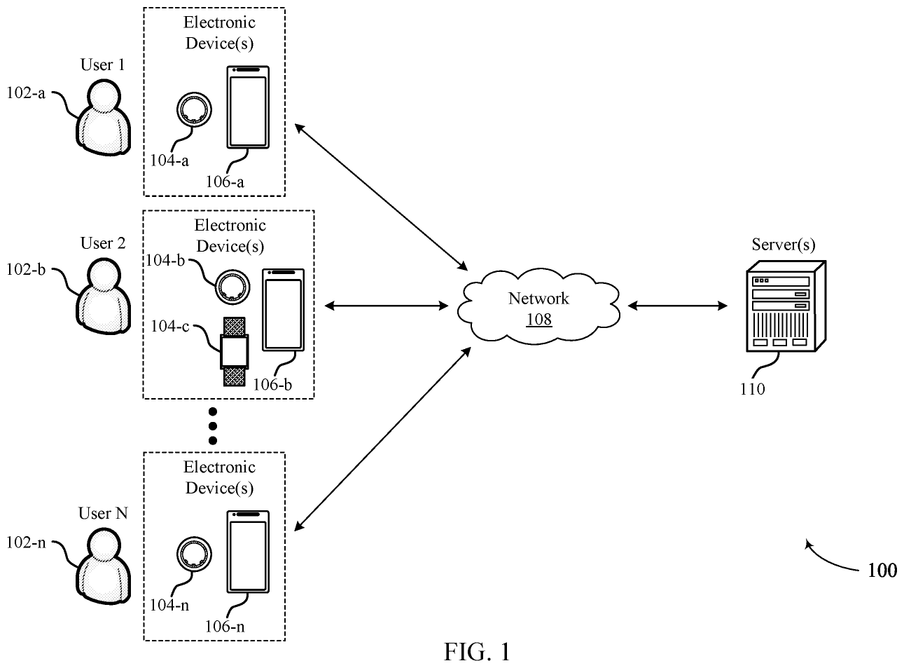

FIG. 1 illustrates an example of a system 100 that supports techniques for blood pressure calibration in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, blood oxygen saturation (SpO2), blood sugar levels (e.g., glucose metrics), and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols. Moreover, in some cases, the wearable device 104 and the user device 106 may be included within (or make up) the same device. For example, in some cases, the wearable device 104 may be configured to execute an application associated with the wearable device 104, and may be configured to display data via a GUI.

In some implementations, the rings 104 (e.g., wearable devices 104) of the system 100 may be configured to collect physiological data from the respective users 102 based on arterial blood flow within the user's finger. In particular, a ring 104 may utilize one or more light-emitting components, such as LEDs (e.g., red LEDs, green LEDs) that emit light on the palm-side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g. in a hypothetical culture with 12 day “weeks,” 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to capture the dynamic physiological baselines of an individual or group of individuals more accurately.

In some aspects, the respective devices of the system 100 may support techniques for calibrating blood pressure measurements acquired by a wearable device 104. For example, the wearable device 104 or a user device 106 may display, via a GUI, instructions for the user 102 to calibrate the blood pressure measurements. The instructions may indicate the user 102 to hold their arm that includes the wearable device 104 at different positions to calibrate the blood pressure measurements. For example, the user 102 may be instructed to hold their arm at a first vertical level above a level of a heart of the user, at a second vertical level below the level of the heart of the user, and at a third level at (e.g., approximately) the level of the heart of the user. As such, the wearable device 104 (e.g., via communications with the user device 106, the network 108, and/or a server 110) may determine a calibration function based on the measurements taken at each level, which may allow the wearable device to adjust future blood pressure measurements for the user 102, as described herein.

It should be appreciated by a person skilled in the art that one or more aspects of the disclosure may be implemented in a system 100 to additionally, or alternatively, solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

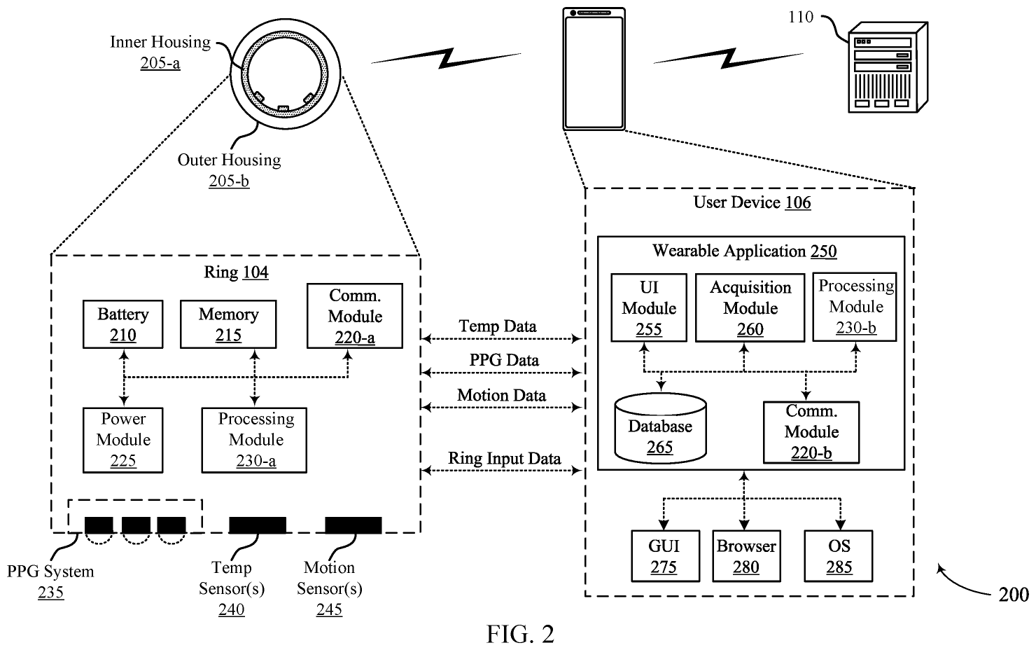

FIG. 2 illustrates an example of a system 200 that supports techniques for blood pressure calibration in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels (SpO2), blood sugar levels (e.g., glucose metrics), and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, PPG data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using adhesives, wraps, clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during charging, and under voltage during discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 where the optical receiver(s) receive transmitted light that is reflected through the region of the user's finger. In some implementations, the PPG system 235 may be configured as a transmissive PPG system 235 where the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BM1160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

In some cases, the wearable device 104 and the user device 106 may be included within (or make up) the same device. For example, in some cases, the wearable device 104 may be configured to execute the wearable application 250, and may be configured to display data via the GUI 275.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the system 200 may support techniques for calibration of blood pressure measurements for a user 102, as described herein. For example, a wearable device 104 (e.g., a ring 104) may use the PPG system 235 to obtain a blood pressure metric while a user 102 holds their arm at different positionings relative to a heart of the user 102. Further, in some cases, the wearable device 104 may analyze how the shape of PPG signals/waveforms (e.g., PPG morphology) changes based on the relative positioning of the user's arm. As such, the wearable device 102 may use the obtained blood pressure metrics and the shape of subsequent PPG signals/waveforms to determine whether a user's arm is raised above or below a level of the user's heart. The wearable device 104 may then predict when the PPG measurements are taken at, above, or below the level of the user's heart, and the wearable device 104 may adjust subsequent blood pressure measurements accordingly, providing more accurate blood pressure metrics to the user 102.

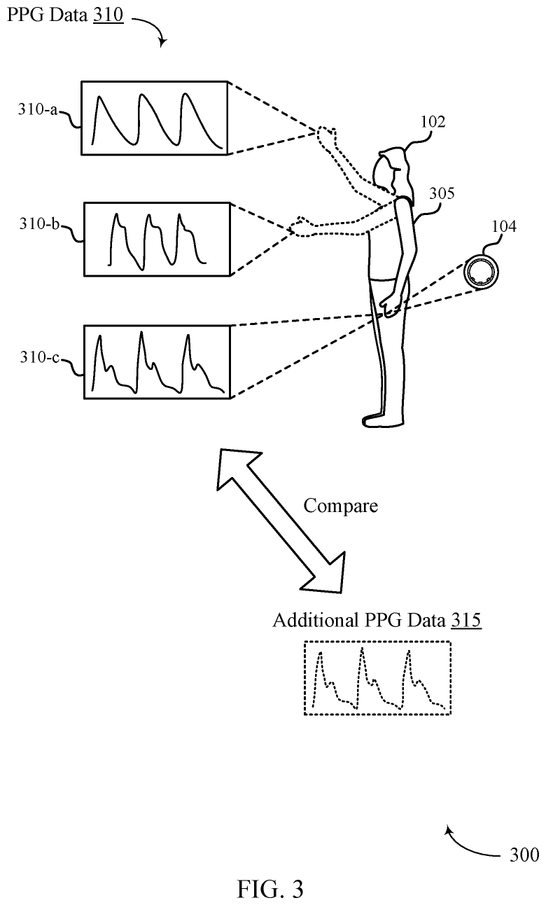

FIG. 3 shows an example of a system 300 that supports techniques for blood pressure calibration in accordance with aspects of the present disclosure. The system 300 illustrates calibration of blood pressure measurements taken by a wearable device 104. While system 300 illustrates a ring wearable device 104, the techniques described herein may be performed using different wearable devices 104, such as a watch wearable device 104, or another device.

The wearable device 104 may be configured to measure blood flow using PPG measurements to obtain a blood pressure metric, which may be indicative of a blood pressure of a user 102. The wearable device 104 may be worn at an arm 305 of the user 102, such as on a finger of the arm 305 (e.g., for a ring wearable device 104) or on a wrist of the arm 305 (e.g., for a watch wearable device 104).

As described previously herein, as the arm 305 of the user 102 moves (e.g., vertically) in relation to a heart of the user, the local blood flow at the arm 305 may be altered due to effects of gravity (e.g., hydrostatic effect) and veno-arterial reflex of the user 102. For example, due to gravity, if the arm 305 is raised above the heart of the user 102, arterial pressure within the arm 305 may decrease, while venous blood flow (e.g., venous return) may increase. Conversely, if the arm 305 is lowered below the level of the heart, the arterial pressure may increase, and venous blood flow may decrease. Meanwhile, the veno-arterial reflex of the user 102 may attempt to regulate blood pressure as the arm 305 moves, which may also affect blood flow. As such, a blood flow observed by the wearable device 104 within the arm 305 may change as the arm 305 moves. This may indicate the wearable device 104 that the blood pressure of the user 102 is changing, even if the blood pressure of the user 102 has remained (e.g., relatively) constant.

Accordingly, to account for movement of the arm 305 relative to the heart of the user 102, the wearable device 104 may perform a calibration procedure. For example, the calibration procedure may include acquiring reference PPG data 310 while the arm 305 (e.g., or another body part at which the wearable device 104) is located in different positions (e.g., at least two different positions) relative to a level of the heart of the user 102. In some examples, the wearable device 104 may obtain PPG data 310-a while the arm is above the level of the heart, PPG data 310-b while the arm is (e.g., approximately) at the level of the heart, and PPG data 310-c while the arm is below the level of the heart (e.g., at the side of the user 102). As such, the wearable device 104 may acquire a respective blood pressure metric based on the PPG data 310-a, the PPG data 310-b, and the PPG data 310-c, corresponding to respective positions of the arm 305.

In some examples, the user 102 may be instructed to place the arm 305 of the user at the different locations, for example, via a GUI of a user device 106 of the user or of the wearable device 104 (e.g., for a watch wearable device 104). For instance, the wearable device 104 may transmit signaling to display instructions, via the GUI, for the user 102 to place the arm 305 at a location below the level of the heart of the user (e.g., by placing the arm 305 by at the side of the user 102), at (e.g., approximately) the level of the heart (e.g., by extending the arm 305 outwards in front of the user 102), above the level of the heart (e.g., by extending the arm 305 upwards), or a combination thereof. In some examples, the instructions may instruct the user 102 to maintain the arm 305 at the respective position for some period of time, which may allow the wearable device 104 to acquire the respective PPG data 310.

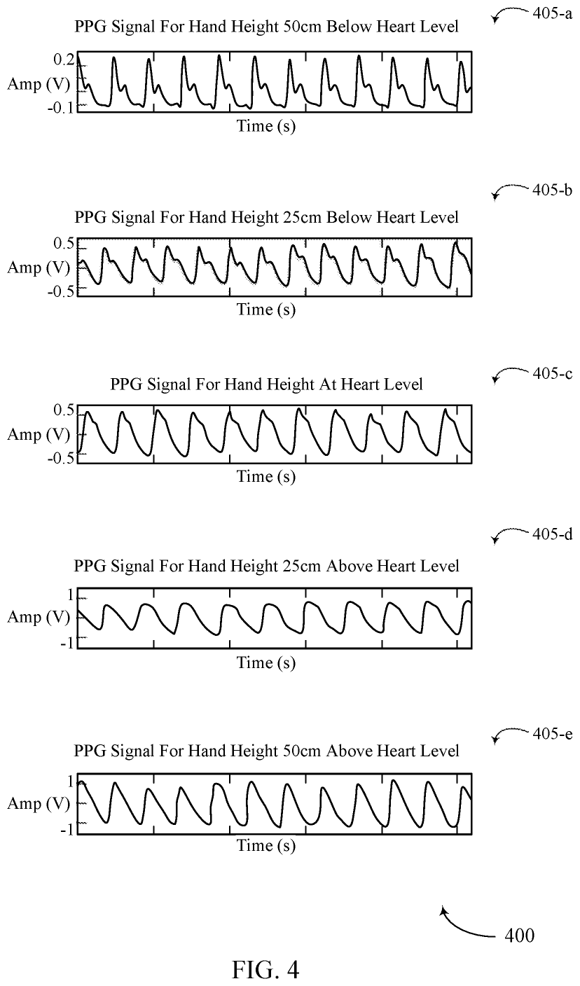

Additionally, or alternatively, the wearable device 104 may determine a position of the arm 305 (e.g., relative to the level of the heart of the user 102) using other factors, such a morphology of collected PPG data 310, acceleration/motion data, and the like. The PPG data 310 may have different morphology depending on a position of the arm 305 relative to the heart, such as different shapes, amplitudes, timings of the PPG data 310. For instance, as shown in FIG. 3, the PPG data 310-a collected while the user's arm 305 is below heart level exhibits a distinct dicrotic notch, where the distinctiveness of the dicrotic notch decreases in the PPG data 310-b and 310-c as the user's arm 305 is raised. As such, the wearable device 104 may determine the position of the arm 305 based on the morphology of the PPG data 310, such as by comparing the morphology of the PPG data to a morphology of reference PPG data 310 (e.g., using one or more machine learning models). The morphology of PPG data 310 relative to the position of the arm 305 is described in more detail with reference to FIG. 4.

Additionally, or alternatively, the wearable device 104 may determine the position of the arm 305 using movement data collected by the wearable device 104 (e.g., using one or more sensors), or based on a known activity of the user 102. For instance, if the wearable device 104 determines the user 102 is asleep, the wearable device 104 may determine that the arm 305 is (e.g., approximately) at the level of the heart of the user 102, as the arm 305 is likely to be laid down by the user 102. By way of another example, the GUI may display instructions for the user to raise their arm 305 from by their side to approximately heart-level, and the system may utilize motion/acceleration data acquired via the wearable device 104 to determine/validate whether the user 102 moved their arm 305 in accordance with the instructions. In this regard, motion/acceleration data collected via wearable device 104 may be used to determine/validate that the user 102 has moved their arm into the correct positions during the calibration procedure.

In some aspects, as part of the calibration procedure, the system may “learn” a PPG morphology of the respective PPG data 310 collected at varying positions. That is, the system may train one or more machine learning models to “learn” or recognize the shape (e.g., PPG morphology, PPG features) of the PPG data 310 collected at different positions of the user's arm 305.

In some examples, the wearable device 104 may determine, as part of the calibration procedure, a calibration function (e.g., an algorithm, a mathematical operation) based on the reference PPG data 310 (e.g., the PPG data 310-a, the PPG data 310-b, and the PPG data 310-c). The calibration function may be used to adjust blood pressure metrics determined using PPG data 310 based on the position of the arm 305 while the PPG data 310 was acquired.

In particular, assuming if the PPG data 310-a, 310-b, and 310-c was collected in a relatively short period of time (e.g., a span of seconds or minutes), it may be assumed that the user's “actual” blood pressure (e.g., blood pressure at the user's heart-level) did not change significantly throughout the calibration procedure. However, as discussed herein, the “observed” blood pressure observed by the wearable device 104 may change as the position of the user's arm 104 changes. In this regard, it may be assumed that each of the PPG data 310-a, 310-b, 310-b waveforms indicate the same “actual” blood pressure for the user, where differences in the PPG data 310 are due to the position of the user's arm 305. In other words, it may be assumed that each of the PPG data 310-a, 310-b, 310-c waveforms are associated with the same “actual” blood pressure, despite the different PPG morphologies. As such, as part of the calibration procedure, the system may utilize a blood pressure metric determined based on the PPG data 310-b collected at heart-level as a “reference” blood pressure metric (e.g., “actual” blood pressure metric), and may determine how to adjust blood pressure metrics determined based on the PPG data 310-a, 310-c to “match” the blood pressure metric determined at heart level. In other words, the calibration function may relate a change in pressure (e.g., in mmHg) of a blood pressure of the user to a position of the arm 305 (e.g., in cm away from the level of the heart), and may be used to adjust the blood pressure metric accordingly. That is, the calibration function may be used to adjust blood pressure metrics that are determined based on PPG data 310 that was not collected at heart-level.

Stated differently, the wearable device 104 may determine the calibration function by adjusting a first blood pressure metric determined based on the PPG data 310-a collected while the arm 305 was above heart level such that the first blood pressure metric matches a “reference” second blood pressure metric determined based on the “reference” PPG data 310-b collected while the arm 305 was at the level of the heart. A similar procedure may be performed using a blood pressure metric determined based on the PPG data 310-c. In some examples, the wearable device 104 may determine multiple calibration functions corresponding to different positions of the arm 305. Alternatively, a single calibration function may account for different positions of the arm 305 (e.g., a parameter for the calibration function may include an indication of the position of the arm 305 relative to the level of the heart).

In some aspects, the determined PPG morphologies of the respective PPG data 310 and the calibration function(s) may be used to calibrate future blood pressure metrics. In other words, the calibration procedure described herein (where PPG data 310 is collected at different positions and used to determine blood pressure metrics) may enable the system to perform more accurate blood pressure metrics for the user in the future. For example, after performing the calibration procedure at a first time, the wearable device 104 may acquire additional PPG data 315 from the user, and may calculate a blood pressure metric for the user based on the additional PPG data 315. In some cases, the position of the user's arm 305 during the time that the additional PPG data 315 was collected may be unknown. As such, using conventional techniques, it may be unknown whether the blood pressure metric determined based on the additional PPG data 315 is approximately equal to the user's actual blood pressure, or if blood pressure metric is artificially higher or lower due to the position of the arm 305.

Accordingly, in some aspects, the system may determine a position of the arm 305 during a time interval corresponding to when the additional PPG data 315 was collected. For example, the wearable device 104 may compare a morphology of the additional PPG data 315 to the morphology of the reference PPG data 310 collected during the calibration procedure (e.g., or other reference PPG data not collected from the user 102). The wearable device 104 (and/or user device 106, servers 110, etc.) may determine the position of the arm 305 based on a match (e.g., a closest match) to the morphology of the additional PPG data 315. In some cases, the wearable device 104 may also determine the position of the arm 305 based on an indication of the position of the arm 305 obtained via the GUI, for example, based on input from the user 102. In other words, in some cases, the user may explicitly indicate the position of the arm 305 while the additional PPG data 315 was collected.

Subsequently, the system may selectively adjust the blood pressure metric determined based on the additional PPG data 315 using one or more calibration functions. The wearable device 104 may determine the calibration function based on the position of the arm. For example, the wearable device 104 may select the calibration function that corresponds to the position of the arm 305 while the additional PPG data 310 was collected from a set of calibration functions. Additionally, or alternatively, the wearable device 104 may input an indication of the position of the arm 305 into the calibration function. For example, the wearable device 104 may input the indication of the position of the arm 305 as a parameter into the calibration function, as well as a measured blood pressure metric, and the calibration function may output an adjusted blood pressure metric. Accordingly, the wearable device 104 may use the calibration function to determine an adjusted blood pressure metric for the user 102 using the additional PPG data 310 and the position of the arm 305.

For example, the system may compare the morphology of the additional PPG data 315 to the morphologies of the baseline PPG data 310-a, 310-b, 310-c, and may therefore determine that the user's arm was likely below heart level at the time the additional PPG data 315 was collected (e.g., by comparing or matching the additional PPG data 315 with the baseline PPG data 310-c). As such, using the calibration function, the blood pressure metric determined based on the additional PPG data 315 may be modified (decreased) due to the fact that observed arterial blood pressure typically increases when the arm 305 is below heart level.

In some cases, the changes in the blood flow at the arm 305 due to the position of the arm 305 may be based on a length of the arm 305. For example, an observed change in the blood pressure may be smaller for a user 102 with a shorter arm 305, relative to a user 102 with a longer arm 305. As such, in some examples, the wearable device 104 may request (e.g., via the GUI) for the user 102 to input a length of the arm 305. Additionally, or alternatively, the wearable device 104 may approximate a length of the arm 305 using a height of the user 102, which may have been previously input by the user 102. The length of the arm 305 may then be used to adjust a blood pressure metric for PPG data collected by the wearable device 104. For example, the calibration function may be adjusted using the length of the arm 305.

In some examples, the wearable device 104 may determine a position of the arm 305 based on one or more pulse transmit times associated with heartbeats of the user 102. For example, the user 102 may wear multiple wearable devices 104 (e.g., on different arms 305 or body parts). At least two wearable devices 104 may detect a pulse corresponding to a heartbeat of the user 102 observed at a respective location of the body of the user 102, and the wearable devices 104 may determine a pulse transmit time between the two respective locations. As such, reference pulse transmit times may be determined by the wearable device 104 corresponding to each location of the arm 305 during the calibration procedure based on the reference PPG data 310 (e.g., first pulse transmit time from heart to hand while user's arm 305 is at heart-level, second pulse transmit time from heart to hand while user's arm 305 is raised, etc.). The wearable device 104 may then determine an additional pulse transmit time during a time period corresponding to the additional PPG data 310, and the wearable device 104 may determine the position of the arm 305 by comparing the additional pulse transmit time to the reference pulse transmit times (e.g., and finding a closest match).

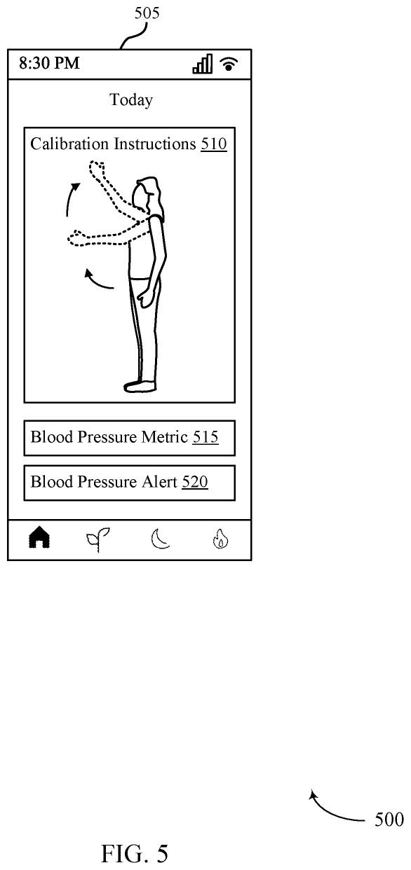

In some examples, the wearable device 104 may transmit an instruction to display a notification of the blood pressure metric at the GUI. For example, the notification of the blood pressure metric may correspond to a numerical representation of the blood pressure metric, such as measurement of systolic pressure and diastolic pressure. Additionally, or alternatively, the notification may correspond to a metric relative to a baseline and may indicate whether the blood pressure of the user 102 is high, average, or low, for example. In some cases, the wearable device 104 may transmit an instruction to display an alert corresponding to the blood pressure metric at the GUI, for instance, if the blood pressure metric is determined to be above a threshold (e.g., corresponding to high blood pressure) or below a threshold (e.g., corresponding to low blood pressure). Displaying information related to the blood pressure metric is described in more detail with reference to FIG. 5.

In some aspects, techniques described herein may also be used to evaluate how the arm 305/hand position of the user 102 relative to the user's heart (due to gravity) affects the “observed” blood pressure in the user's arm 305/hand. In particular, the system may be configured to evaluate PPG morphology of the additional PPG data 315 to evaluate how the PPG morphology appears when the user's blood pressure is lower or higher than normal. For example, as noted previously herein, if the user's arm 305 is A cm higher than heart-level, the blood pressure at the user's hand may drop by B mmHg. Conversely, if the user's arm 305 is C cm lower than heart-level, the blood pressure at the user's hand may increase by D mmHg. In this regard, if the system collects the additional PPG data 315 at heart level, but observes that the additional PPG data 315 exhibits similar PPG morphology as the baseline PPG data 310-c collected below heart-level (e.g., C cm lower than heart-level), the system may be configured estimate that the user's blood pressure (as determined by the additional PPG data 315) is D mmHg higher than normally. In other words, if the position of the arm 305 is known during the time that the additional PPG data 315 is collected, this arm position data may also be leveraged to more accurately determine the user's blood pressure by comparing the morphology of the additional PPG data 315 to the respective baseline PPG data 310-a, 310-b, 310-c.

Accordingly, the wearable device 104 may perform a calibration procedure to adjust a blood pressure metric corresponding to a blood pressure of a user 102, improving the accuracy of health insights provided to the user 102.

FIG. 4 shows an example of PPG diagrams 400 that support blood pressure calibration in accordance with aspects of the present disclosure. The PPG diagrams 400 illustrate various PPG data 405 acquired by a wearable device 104 worn on an arm of a user 102. Each of the PPG data 405 may correspond to data collected by the wearable device 104 while the arm of the user 102 was located at a respective position relative to a level of a heart of the user 102.

For example, PPG data 405-a may correspond to a PPG signal when a hand of the user 102 is 50 cm below the level of the heart, and PPG data 405-b may correspond to a PPG signal when the hand of the user is 25 cm below the level of the heart. PPG data 405-c may correspond to a PPG signal when the hand is (e.g., approximately) at the level of the heart, PPG data 405-d may correspond to a PPG signal when the hand is 25 cm above the level of the heart, and PPG data 405-e may correspond to a PPG signal when the hand is 50 cm above the level of the heart.

In some examples, the PPG data 405-a, the PPG data 405-b, the PPG data 405-c, the PPG data 405-d, and the PPG data 405-e may correspond to reference PPG data. For example, the reference PPG data may be collected by the wearable device from the user 102 during a calibration procedure, as described herein. In some examples, the wearable device 104 may instruct (e.g., via a GUI) the user 102 to manipulate the arm of the user 102 to a respective location to collect respective PPG data 405. Additionally, or alternatively, the reference PPG data may be baseline data common for multiple users 102. While FIG. 4 illustrates reference PPG data for five different locations of the arm of the user 102 relative to the level of the heart, the wearable device 104 may collect reference PPG data at a more or fewer (e.g., three) locations relative to the level of the heart of the user 102.

The PPG data 405 may each exhibit different morphology (e.g., different shapes, different features) depending on a position of the arm relative to the heart of the user. For example, the PPG data 405 may include different timings between systolic peaks and diastolic peaks, different shapes of the systolic peaks or the diastolic peaks, different amplitudes of the systolic peaks or the diastolic peaks, different shapes of the first derivative of the corresponding PPG signal, different shapes of the second derivative of the corresponding PPG signal, different timings between peaks of the first derivative or the second derivative of the PPG signal, or any combination thereof, based on the position of the arm. Each of these respective PPG morphological features may be used to estimate the position of the user's hand/arm, and/or adjust blood pressure metrics.

For instance, as the relative height of the arm decreases (e.g., from the PPG data 405-e to the PPG data 405-a), the amplitude (Amp) of the PPG signal may decrease, which may be measured in Volts by the wearable device 104. Additionally, or alternatively, a notch in the PPG signal (e.g., corresponding to a dicrotic notch) may become more prominent in the PPG data 405 as the relative height of the arm decreases, and as the relative height of the arm increases, the notch may become less prominent and the PPG signal may appear more triangular. The timings between peaks in the PPG data 405, or between peaks of the first derivative or the second derivative of the PPG signal, may also change based on the relative height of the arm of the user 102.

As described herein, the wearable device may use the reference PPG data to determine a position of the arm of the user 102. For example, the wearable device 104 (e.g., directly or via one or more other devices as described herein) may compare morphological features of additional PPG data acquired from the user 102 during a time period to morphological features of the reference PPG data collected during a calibration period. For instance, the wearable device 104 may compare the additional PPG data to the reference PPG data, and the wearable device 104 may determine a set of PPG data 405 that has a (e.g., closest) matching morphology to the morphology of the additional PPG data 405. As such, the wearable device 104 may determine the position of the arm of the user 102, and the wearable device may adjust a blood pressure calculated from the additional PPG data based on the position of the arm (e.g., using a calibration function, as described herein with reference to FIG. 3).

Additionally, or alternatively, to determine the position of the arm of the user 102 based on the reference PPG data, the wearable device 104 (e.g., directly or via the user device 106 or a server 110) may use one or more machine learning models (e.g., machine learning classifiers). For example, the wearable device 104 (e.g., or another device) may input the reference PPG data to the one or more machine learning models and extract one or more sets of reference PPG features corresponding to different positions of the arm of the user. The wearable device 104 may then input the additional PPG data to the one or more machine learning models to extract features corresponding to the additional PPG data. The wearable device may compare the features corresponding to the additional PPG data to the one or more sets of reference PPG features to determine the position of the arm of the user 102 (e.g., by finding a closest match).

Accordingly, the wearable device 104 may determine the position of an arm of the user 102 using the reference PPG data, allowing the wearable device 104 to adjust a blood pressure metric to provide more accurate blood pressure information.

FIG. 5 shows an example of a GUI 500 that supports techniques for blood pressure calibration in accordance with aspects of the present disclosure. The GUI 500 may be displayed as part of a user device 106 of a user 102, or as part of a wearable device 104 (e.g., a watch wearable device 104). In some examples, the GUI 500 may receive instructions from a device, such as the wearable device 104 or a server 110, to display information to the user 102. The GUI 500 illustrates an application page 505 that includes such information. However, the information displayed on the application added to the application page 505.

In some examples, the application page 505 may display calibration instructions 510. The calibration instruction 510 may instruct the user 102 to move an arm of the user 102 to certain positions for some time duration. As such, the wearable device 104 may collect PPG data at each position for the time duration, allowing the wearable device 104 to collect reference PPG data. The reference PPG data may be associated with different positions of the arm of the user relative to a (e.g., vertical) level of the heart of the user 102, and the reference PPG data may be used to adjust a blood pressure metric based on additional PPG data collected by the wearable device 104 as described herein.

In some cases, the wearable device 104 may acquire motion data during the calibration procedure. For example, the wearable device 104 may collect motion data after instructing the user 102 to move the arm of the user 102. As such, the wearable device 104 may determine whether the user 102 moved the arm in accordance with the instructions, and the wearable device 104 may collect the reference PPG data after the user 102 has moved the arm.

After the wearable device 104 collects the additional PPG data, the application page 505 may display a notification of a blood pressure metric 515 adjusted based on the reference PPG data. The blood pressure metric 515 may be a numerical representation of the blood pressure metric, such as measurement of systolic pressure over diastolic pressure. Additionally, or alternatively, the blood pressure metric 515 may correspond to a metric relative to a baseline blood pressure, and the blood pressure metric 515 may indicate whether the blood pressure of the user 102 based on the additional PPG data is high, average, or low, for example, relative to the baseline blood pressure.

In some cases, the wearable device 104 may transmit an instruction to cause the application page 505 to display a blood pressure alert 520. For example, the wearable device 104 (e.g., or the user device 106, or another device) may determine that a blood pressure of the user 102 may be too high (e.g., above a threshold value), or too low (e.g., below a threshold value) based on the blood pressure metric (e.g., based on the blood pressure metric being above or below a threshold). As such, the application pressure of the user 102 may be too high or too low. In some examples, the application page 505 may display instructions for the user 102 to cause the blood pressure of the user 102 to no longer satisfy one or more thresholds (e.g., to lower or raise the blood pressure of the user 102). Additionally, or alternatively, the application page 505 may display an instruction for the user 102 to seek medical attention due to the blood pressure of the user 102.

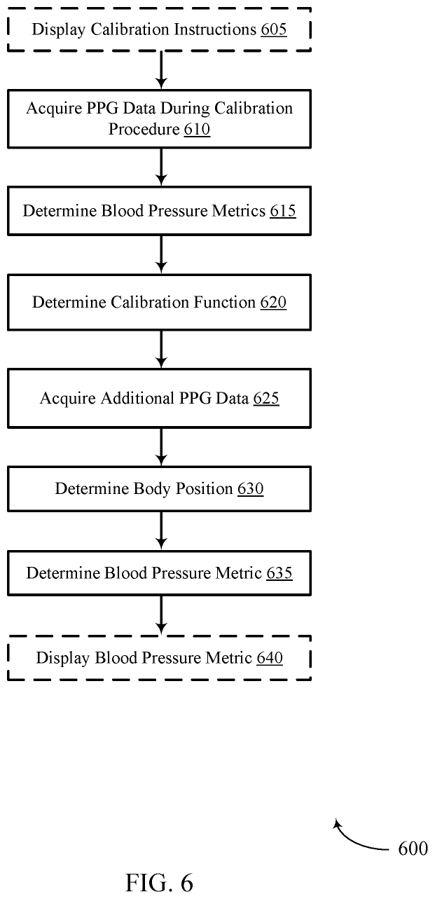

FIG. 6 shows an example of a flowchart 600 that supports techniques for blood pressure calibration in accordance with aspects of the present disclosure. Aspects of the flowchart 600 may implement, or be implemented by, aspects of the system 100, the system 200, the system 300, or any combination thereof. In some examples, the steps of the flowchart 600 may be performed in a different order than shown. Additionally, or alternatively, some steps may be added to the flowchart 600, and some steps may be omitted or optional. The flowchart 600 may illustrate steps for calibrating blood pressure measurements for determining a blood pressure metric based on PPG collected by a wearable device 104.

At 605, a GUI (e.g., of the wearable device 104 or a user device 106) may display calibration instructions. For example, the GUI may instruct a user 102 to place a body part of the user (e.g., in which the wearable device 104 is located, such as a hand, wrist, or arm) at a first location below the level of the heart of the user (e.g., by placing the body part by at the side of the user 102) for some time duration, at a second location at (e.g., approximately) the level of the heart (e.g., by extending the arm 305 outwards in front of the user 102) for some time duration, and at a third location above the level of the heart (e.g., by extending the body part upwards) for some time duration.

At 610, the wearable device 104 may acquire reference PPG data during the calibration procedure. For example, the wearable device 104 may acquire first PPG data at the first location, second PPG data at the second location, and third PPG data at the third location based on the GUI providing the instructions to the user 102. In some examples, the first PPG data, the second PPG data, and the third PPG data may be associated with a first PPG morphology, a second PPG morphology, and a third PPG morphology, respectively. The PPG morphologies may be associated with one or more features of a respective PPG signal.

At 615, the wearable device 104 (e.g., or another device) may determine, as part of the calibration procedure, one or more blood pressure metrics associated with the reference PPG data. For example, the wearable device 104 may determine a first blood pressure metric, a second blood pressure metric, and a third blood pressure metric based on the first PPG data, the second PPG data, and the third PPG data, respectively.

At 620, the wearable device 104 (e.g., or another device) may determine, as part of the calibration procedure, a calibration function associated with blood pressure metrics determined according to reference PPG data. For example, the calibration function may be based on a comparison between the second blood pressure metric (e.g., associated with PPG data measured at the level of the heart) and the first blood pressure metric, and a comparison of the second blood pressure metric and the third blood pressure metric. In some examples, the calibration function may be based on a comparison of the second PPG morphology (e.g., associated with PPG data measured at the level of the heart) and the first PPG morphology, and a comparison of the second PPG morphology and the third PPG morphology.

At 625, the wearable device 104 may acquire additional PPG data from the user during an additional time interval. The additional PPG data may be associated with an additional PPG morphology, which may include one or more features of a PPG signal associated with the PPG data.

At 630, the wearable device 104 may determine a position of the body part of the user during the additional time interval based on a comparison between the additional PPG morphology and the first PPG morphology, the second PPG morphology, the third PPG morphology, or a combination thereof. For example, the wearable device 104 may find a closest match between the additional PPG morphology and the first PPG morphology, the second PPG morphology, or the third PPG morphology.

At 635, the wearable device 104 may determine a blood pressure metric for the user during the additional time interval based on the additional PPG data. In some examples, the blood pressure metric may be based on the determined position of the body part using the additional PPG morphology. Additionally, or alternatively, the blood pressure metric may be based on the calibration function determined by the wearable device 104.

At 640, the GUI may display an indication of the blood pressure metric based on determining the blood pressure metric. Accordingly, the user 102 may obtain information relating to a blood pressure of the user 102 with higher accuracy based on the calibration procedure.

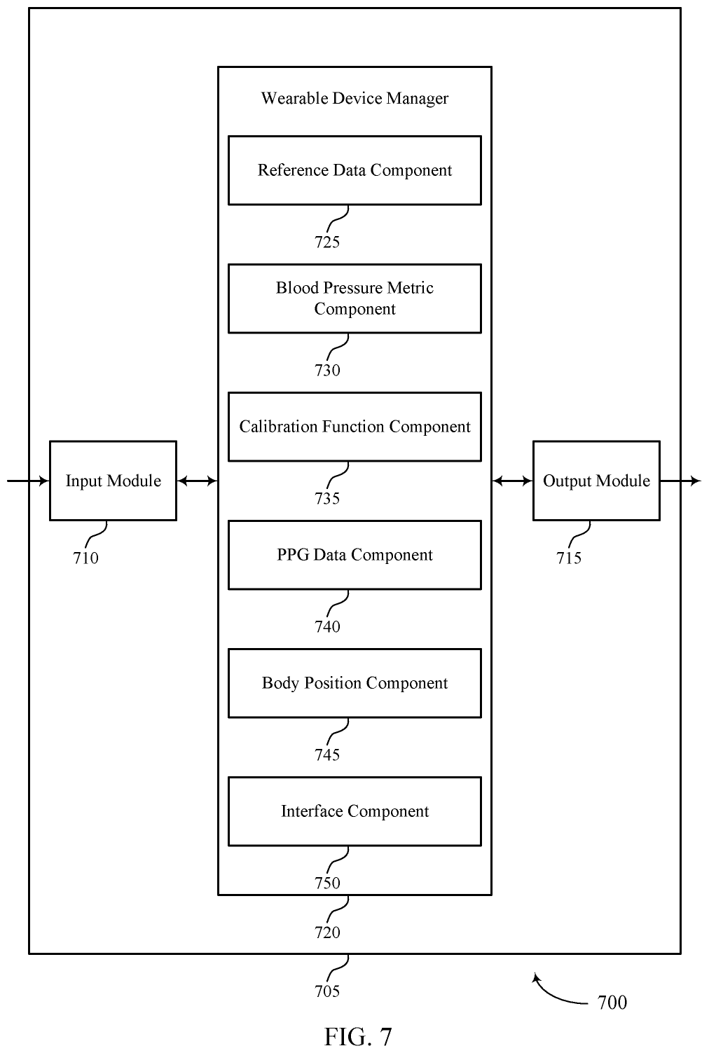

FIG. 7 shows a block diagram 700 of a device 705 that supports techniques for blood pressure calibration in accordance with aspects of the present disclosure. The device 705 may include an input module 710, an output module 715, and a wearable device manager 720. The device 705, or one of more components of the device 705 (e.g., the input module 710, the output module 715, the wearable device manager 720), may include at least one processor, which may be coupled with at least one memory, to support the described techniques. Each of these components may be in communication with one another (e.g., via one or more buses).

For example, the wearable device manager 720 may include a reference data component 725, a blood pressure metric component 730, a calibration function component 735, a PPG data component 740, a body position component 745, an interface component 750, or any combination thereof. In some examples, the wearable device manager 720, or various components thereof, may be configured to perform various operations (e.g., receiving, monitoring, transmitting) using or otherwise in cooperation with the input module 710, the output module 715, or both. For example, the wearable device manager 720 may receive information from the input module 710, send information to the output module 715, or be integrated in combination with the input module 710, the output module 715, or both to receive information, transmit information, or perform various other operations as described herein.

The wearable device manager 720 may support calibrating blood pressure measurements performed in accordance with examples as disclosed herein. The reference data component 725 may be configured as or otherwise support a means for acquiring, during a calibration procedure, reference PPG data from a body part of a user using a wearable device, the reference PPG data comprising first PPG data acquired during a first time interval that the body part is in a first position that is at approximately a level of a heart of the user, and second PPG data acquired during a second time interval that the body part is in a second position that is above or below the level of the heart of the user, wherein the first PPG data and the second PPG data are associated with a first PPG morphology and a second PPG morphology, respectively. The blood pressure metric component 730 may be configured as or otherwise support a means for determining, as part of the calibration procedure and based on the reference PPG data, a first blood pressure metric and a second blood pressure metric associated with the first position and the second position, respectively. The calibration function component 735 may be configured as or otherwise support a means for determining, as part of the calibration procedure, a calibration function associated with blood pressure metrics determined according to PPG data collected at a plurality of positions based at least in part on a comparison between the first blood pressure metric and the second blood pressure metric. The PPG data component 740 may be configured as or otherwise support a means for acquiring additional PPG data from the body part of the user using the wearable device during an additional time interval, the additional PPG data associated with an additional PPG morphology. The body position component 745 may be configured as or otherwise support a means for determining a position of the body part of the user during the additional time interval based at least in part on a comparison between the additional PPG morphology and the first PPG morphology, the second PPG morphology, or both. The blood pressure metric component 730 may be configured as or otherwise support a means for determining a blood pressure metric for the user during the additional time interval based at least in part on the additional PPG data, the determined position of the body part, and the calibration function. The interface component 750 may be configured as or otherwise support a means for transmitting an instruction to a user interface associated with the wearable device, a user device, or both, the instruction configured to cause the user interface to display a notification of the blood pressure metric.

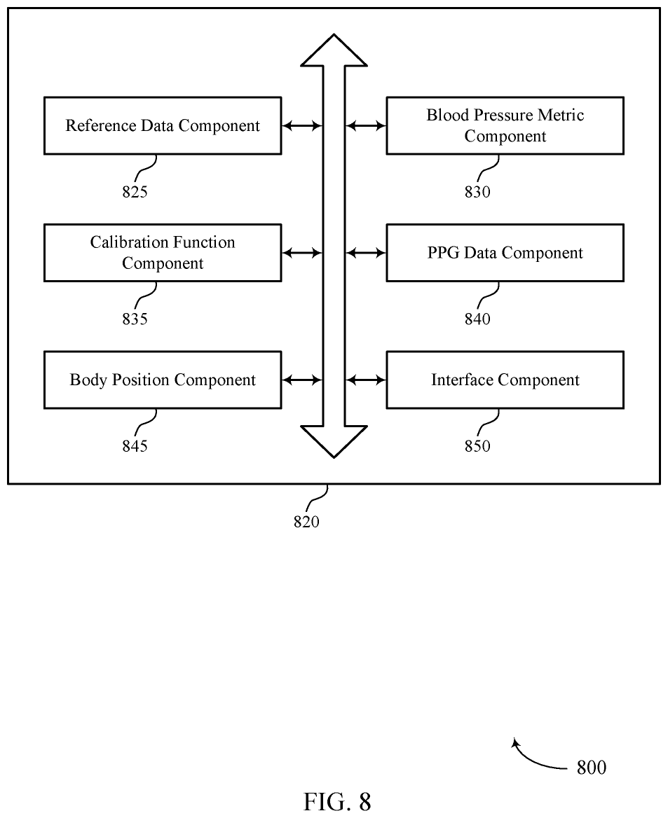

FIG. 8 shows a block diagram 800 of a wearable device manager 820 that supports techniques for blood pressure calibration in accordance with aspects of the present disclosure. The wearable device manager 820 may be an example of aspects of a wearable device manager or a wearable device manager 720, or both, as described herein. The wearable device manager 820, or various components thereof, may be an example of means for performing various aspects of blood pressure calibration as described herein. For example, the wearable device manager 820 may include a reference data component 825, a blood pressure metric component 830, a calibration function component 835, a PPG data component 840, a body position component 845, an interface component 850, or any combination thereof. Each of these components, or components of subcomponents thereof (e.g., one or more processors, one or more memories), may communicate, directly or indirectly, with one another (e.g., via one or more buses).

The wearable device manager 820 may support calibrating blood pressure measurements performed in accordance with examples as disclosed herein. The reference data component 825 may be configured as or otherwise support a means for acquiring, during a calibration procedure, reference PPG data from a body part of a user using a wearable device, the reference PPG data comprising first PPG data acquired during a first time interval that the body part is in a first position that is at approximately a level of a heart of the user, and second PPG data acquired during a second time interval that the body part is in a second position that is above or below the level of the heart of the user, wherein the first PPG data and the second PPG data are associated with a first PPG morphology and a second PPG morphology, respectively. The blood pressure metric component 830 may be configured as or otherwise support a means for determining, as part of the calibration procedure and based on the reference PPG data, a first blood pressure metric and a second blood pressure metric associated with the first position and the second position, respectively. The calibration function component 835 may be configured as or otherwise support a means for determining, as part of the calibration procedure, a calibration function associated with blood pressure metrics determined according to PPG data collected at a plurality of positions based at least in part on a comparison between the first blood pressure metric and the second blood pressure metric. The PPG data component 840 may be configured as or otherwise support a means for acquiring additional PPG data from the body part of the user using the wearable device during an additional time interval, the additional PPG data associated with an additional PPG morphology. The body position component 845 may be configured as or otherwise support a means for determining a position of the body part of the user during the additional time interval based at least in part on a comparison between the additional PPG morphology and the first PPG morphology, the second PPG morphology, or both. In some examples, the blood pressure metric component 830 may be configured as or otherwise support a means for determining a blood pressure metric for the user during the additional time interval based at least in part on the additional PPG data, the determined position of the body part, and the calibration function. The interface component 850 may be configured as or otherwise support a means for transmitting an instruction to a user interface associated with the wearable device, a user device, or both, the instruction configured to cause the user interface to display a notification of the blood pressure metric.

In some examples, the blood pressure metric component 830 may be configured as or otherwise support a means for adjusting the second blood pressure metric by an amount to match to the reference blood pressure metric, wherein determining the calibration function is based at least in part on the amount that the second blood pressure metric is adjusted by to match the reference blood pressure metric.

In some examples, the first blood pressure metric associated with the first position that is at approximately the level of the heart of the user is used as a reference blood pressure metric. In some examples, the calibration function comprises an algorithm or mathematical operation used to adjust blood pressure metrics collected at positions other than the first position relative to the reference blood pressure metric.

In some examples, to support acquiring the reference PPG data during the calibration procedure, the reference data component 825 may be configured as or otherwise support a means for acquiring third PPG data that is acquired from the body part of the user during a third time interval that the body part is in a third position that is below the level of the heart of the user, wherein the third PPG data is associated with a third PPG morphology, and wherein the blood pressure metric is determined based at least in part on a comparison between the additional PPG morphology and the third PPG morphology.

In some examples, the body part may include a hand or a finger of an arm of the user, and the calibration function component 835 may be configured as or otherwise support a means for receiving a user input indicating a length of the arm of the user, a height of the user, or both, wherein determining the calibration function, determining the blood pressure metric, or both, is based at least in part on the length of the arm of the user, an estimation of the length of the arm of the user performed based on the height of the user, or both.

In some examples, to support determining the blood pressure metric for the additional time interval, the blood pressure metric component 830 may be configured as or otherwise support a means for determining an initial blood pressure metric based at least in part on the additional PPG data collected during the additional time interval. In some examples, to support determining the blood pressure metric for the additional time interval, the blood pressure metric component 830 may be configured as or otherwise support a means for adjusting the initial blood pressure metric to determine the blood pressure metric based at least in part on the determined position of the body part and the calibration function.

In some examples, to support determining the position of the body part during the additional time interval, the body position component 845 may be configured as or otherwise support a means for inputting the first PPG data and the second PPG data into a machine learning model. In some examples, to support determining the position of the body part during the additional time interval, the body position component 845 may be configured as or otherwise support a means for extracting, using the machine learning model, a first set of reference PPG features and a second set of reference PPG features from the first PPG data and the second PPG data, respectively. In some examples, to support determining the position of the body part during the additional time interval, the body position component 845 may be configured as or otherwise support a means for inputting the additional PPG data into a machine learning classifier. In some examples, to support determining the position of the body part during the additional time interval, the body position component 845 may be configured as or otherwise support a means for extracting, using the machine learning model, an additional set of PPG features from the additional PPG data. In some examples, to support determining the position of the body part during the additional time interval, the body position component 845 may be configured as or otherwise support a means for determining the position of the body part during the additional time interval based at least in part on a comparison between the additional set of PPG features and the first and second sets of reference PPG features.

In some examples, the position of the body part is determined based at least in part on a comparison between respective sets of PPG features associated with the first PPG morphology, the second PPG morphology, and the additional PPG morphology. In some examples, the respective sets of PPG features comprise a relative timing between systolic peaks and diastolic peaks, an amplitude of systolic peaks, an amplitude of diastolic peaks, a shape of a first derivative of the first PPG data, the second PPG data, the additional PPG data, or any combination thereof, a shape of a second derivative of the first PPG data, the second PPG data, the additional PPG data, or any combination thereof, a timing between peaks of the first derivative or the second derivative, or any combination thereof.

In some examples, the interface component 850 may be configured as or otherwise support a means for transmitting one or more signals to the wearable device, the user device, or both, wherein the one or more signals are configured to cause the user interface to display a set of instructions for performing the calibration procedure, wherein the set of instructions displayed by the user interface comprise a first instruction for the user to maintain the body part in the first position for the first time interval, and a second instruction for the user to maintain the body part in the second position for the second time interval.

In some examples, the reference data component 825 may be configured as or otherwise support a means for acquiring motion data from one or more motion sensors of the wearable device based at least in part on transmitting the one or more signals to the wearable device. In some examples, the reference data component 825 may be configured as or otherwise support a means for confirming that the user manipulated the body part in accordance with the set of instructions displayed by the user interface based at least in part on the motion data, wherein acquiring the reference PPG data, determining the calibration function, or both, is based at least in part on confirming that the user manipulated the body part in accordance with the set of instructions.

In some examples, the body position component 845 may be configured as or otherwise support a means for receiving, via the user interface, an indication of a relative position of the body part of the user during the additional time interval, wherein the position is determined based at least in part on the indication of the relative position.

In some examples, the blood pressure metric component 830 may be configured as or otherwise support a means for determining, as part of the calibration procedure and based at least in part on the reference PPG data, a first pulse transmit time associated with a first heartbeat observed at the body part in the first position, and a second pulse transmit time associated with a second heartbeat observed at the body part in the second position, wherein the first blood pressure metric and the second blood pressure metric are based at least in part on the first pulse transmit time and the second pulse transmit time, respectively. In some examples, the blood pressure metric component 830 may be configured as or otherwise support a means for determining, based at least in part on the additional PPG data, an additional pulse transmit time associated with an additional heartbeat observed at the body part during the additional time interval, wherein the position of the body part of the user during the additional time interval is further based on a comparison of the additional pulse transmit time with the first pulse transmit time, the second pulse transmit time, or both.

In some examples, each of the first PPG data, the second PPG data, and the additional PPG data are associated with a plurality of wavelengths. In some examples, the first PPG morphology, the second PPG morphology, and the additional PPG morphology are associated with PPG features of the plurality of wavelengths.

In some examples, the wearable device comprises a wearable ring device. In some examples, the body part comprises a finger of the user. In some examples, the wearable device comprises a wrist-worn wearable device. In some examples, the body part comprises a wrist of the user.

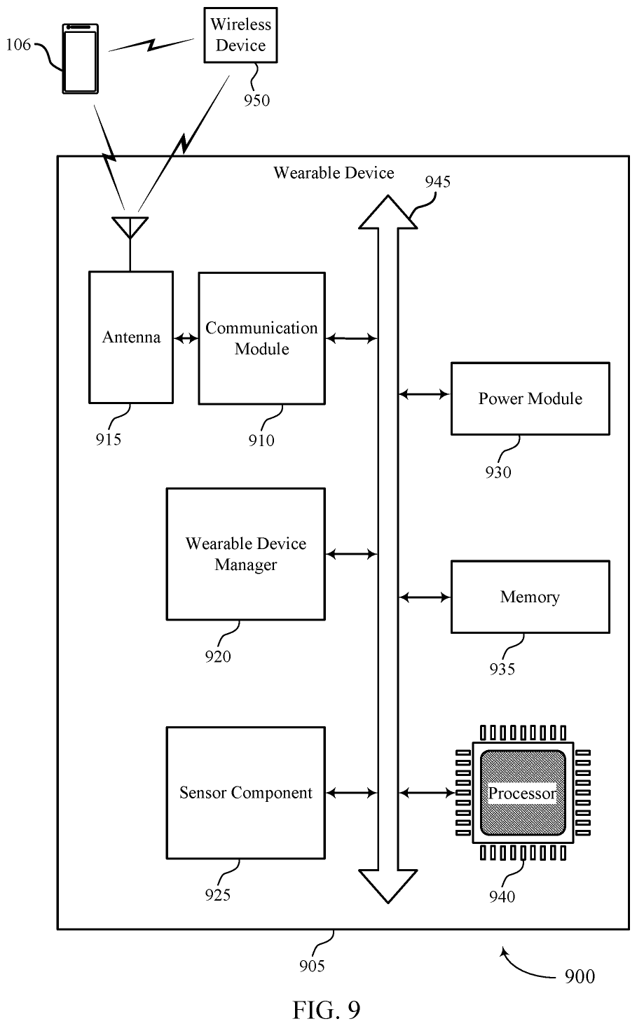

FIG. 9 shows a diagram of a system 900 including a device 905 that supports techniques for blood pressure calibration in accordance with aspects of the present disclosure. The device 905 may be an example of or include components of a device 705 as described herein. The device 905 may include an example of a wearable device 104, as described previously herein. The device 905 may include components for bi-directional communications including components for transmitting and receiving communications with a user device 106 and a server 110, such as a wearable device manager 920, a communication module 910, one or more antennas 915, a sensor component 925, a power module 930, at least one memory 935, at least one processor 940, and a wireless device 950. These components may be in electronic communication or otherwise coupled (e.g., operatively, communicatively, functionally, electronically, electrically) via one or more buses (e.g., a bus 945).

The wearable device manager 920 may support calibrating blood pressure measurements performed in accordance with examples as disclosed herein. For example, the wearable device manager 920 may be configured as or otherwise support a means for acquiring, during a calibration procedure, reference PPG data from a body part of a user using a wearable device, the reference PPG data comprising first PPG data acquired during a first time interval that the body part is in a first position that is at approximately a level of a heart of the user, and second PPG data acquired during a second time interval that the body part is in a second position that is above or below the level of the heart of the user, wherein the first PPG data and the second PPG data are associated with a first PPG morphology and a second PPG morphology, respectively. The wearable device manager 920 may be configured as or otherwise support a means for determining, as part of the calibration procedure and based on the reference PPG data, a first blood pressure metric and a second blood pressure metric associated with the first position and the second position, respectively. The wearable device manager 920 may be configured as or otherwise support a means for determining, as part of the calibration procedure, a calibration function associated with blood pressure metrics determined according to PPG data collected at a plurality of positions based at least in part on a comparison between the first blood pressure metric and the second blood pressure metric. The wearable device manager 920 may be configured as or otherwise support a means for acquiring additional PPG data from the body part of the user using the wearable device during an additional time interval, the additional PPG data associated with an additional PPG morphology. The wearable device manager 920 may be configured as or otherwise support a means for determining a position of the body part of the user during the additional time interval based at least in part on a comparison between the additional PPG morphology and the first PPG morphology, the second PPG morphology, or both. The wearable device manager 920 may be configured as or otherwise support a means for determining a blood pressure metric for the user during the additional time interval based at least in part on the additional PPG data, the determined position of the body part, and the calibration function. The wearable device manager 920 may be configured as or otherwise support a means for transmitting an instruction to a user interface associated with the wearable device, a user device, or both, the instruction configured to cause the user interface to display a notification of the blood pressure metric.

By including or configuring the wearable device manager 920 in accordance with examples as described herein, the device 905 may support techniques for calibration of blood pressure measurements, which may allow for a wearable device to measure blood pressure of a user with increased accuracy.

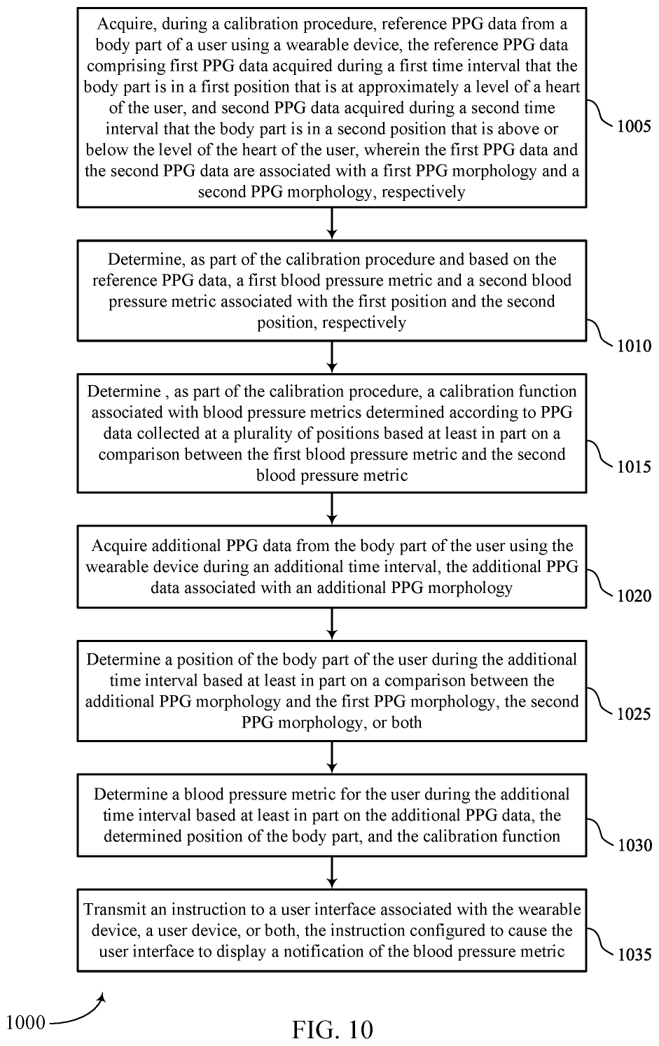

FIG. 10 shows a flowchart illustrating a method 1000 that supports techniques for blood pressure calibration in accordance with aspects of the present disclosure. The operations of the method 1000 may be implemented by a wearable device or its components as described herein. For example, the operations of the method 1000 may be performed by a wearable device as described with reference to FIGS. 1 through 9. In some examples, a wearable device may execute a set of instructions to control the functional elements of the wearable device to perform the described functions. Additionally, or alternatively, the wearable device may perform aspects of the described functions using special-purpose hardware.

At 1005, the method may include acquiring, during a calibration procedure, reference PPG data from a body part of a user using a wearable device, the reference PPG data comprising first PPG data acquired during a first time interval that the body part is in a first position that is at approximately a level of a heart of the user, and second PPG data acquired during a second time interval that the body part is in a second position that is above or below the level of the heart of the user, wherein the first PPG data and the second PPG data are associated with a first PPG morphology and a second PPG morphology, respectively. The operations of 1005 may be performed in accordance with examples as disclosed herein. In some examples, aspects of the operations of 1005 may be performed by a reference data component 825 as described with reference to FIG. 8.

At 1010, the method may include determining, as part of the calibration procedure and based on the reference PPG data, a first blood pressure metric and a second blood pressure metric associated with the first position and the second position, respectively. The operations of 1010 may be performed in accordance with examples as disclosed herein. In some examples, aspects of the operations of 1010 may be performed by a blood pressure metric component 830 as described with reference to FIG. 8.

At 1015, the method may include determining, as part of the calibration procedure, a calibration function associated with blood pressure metrics determined according to PPG data collected at a plurality of positions based at least in part on a comparison between the first blood pressure metric and the second blood pressure metric. The operations of 1015 may be performed in accordance with examples as disclosed herein. In some examples, aspects of the operations of 1015 may be performed by a calibration function component 835 as described with reference to FIG. 8.

At 1020, the method may include acquiring additional PPG data from the body part of the user using the wearable device during an additional time interval, the additional PPG data associated with an additional PPG morphology. The operations of 1020 may be performed in accordance with examples as disclosed herein. In some examples, aspects of the operations of 1020 may be performed by a PPG data component 840 as described with reference to FIG. 8.

At 1025, the method may include determining a position of the body part of the user during the additional time interval based at least in part on a comparison between the additional PPG morphology and the first PPG morphology, the second PPG morphology, or both. The operations of 1025 may be performed in accordance with examples as disclosed herein. In some examples, aspects of the operations of 1025 may be performed by a body position component 845 as described with reference to FIG. 8.

At 1030, the method may include determining a blood pressure metric for the user during the additional time interval based at least in part on the additional PPG data, the determined position of the body part, and the calibration function. The operations of 1030 may be performed in accordance with examples as disclosed herein. In some examples, aspects of the operations of 1030 may be performed by a blood pressure metric component 830 as described with reference to FIG. 8.

At 1035, the method may include transmitting an instruction to a user interface associated with the wearable device, a user device, or both, the instruction configured to cause the user interface to display a notification of the blood pressure metric. The operations of 1035 may be performed in accordance with examples as disclosed herein. In some examples, aspects of the operations of 1035 may be performed by an interface component 850 as described with reference to FIG. 8.

It should be noted that the methods described above describe possible implementations, and that the operations and the steps may be rearranged or otherwise modified and that other implementations are possible. Furthermore, aspects from two or more of the methods may be combined.

A method for calibrating blood pressure measurements performed by a wearable device is described. The method may include acquiring, during a calibration procedure, reference PPG data from a body part of a user using a wearable device, the reference PPG data comprising first PPG data acquired during a first time interval that the body part is in a first position that is at approximately a level of a heart of the user, and second PPG data acquired during a second time interval that the body part is in a second position that is above or below the level of the heart of the user, wherein the first PPG data and the second PPG data are associated with a first PPG morphology and a second PPG morphology, respectively, determining, as part of the calibration procedure and based on the reference PPG data, a first blood pressure metric and a second blood pressure metric associated with the first position and the second position, respectively, determining, as part of the calibration procedure, a calibration function associated with blood pressure metrics determined according to PPG data collected at a plurality of positions based at least in part on a comparison between the first blood pressure metric and the second blood pressure metric, acquiring additional PPG data from the body part of the user using the wearable device during an additional time interval, the additional PPG data associated with an additional PPG morphology, determining a position of the body part of the user during the additional time interval based at least in part on a comparison between the additional PPG morphology and the first PPG morphology, the second PPG morphology, or both, determining a blood pressure metric for the user during the additional time interval based at least in part on the additional PPG data, the determined position of the body part, and the calibration function, and transmitting an instruction to a user interface associated with the wearable device, a user device, or both, the instruction configured to cause the user interface to display a notification of the blood pressure metric.

A wearable device for calibrating blood pressure measurements performed is described. The wearable device may include one or more memories storing processor-executable code, and one or more processors coupled with the one or more memories. The one or more processors may individually or collectively be operable to execute the code to cause the wearable device to acquire, during a calibration procedure, reference PPG data from a body part of a user using a wearable device, the reference PPG data comprising first PPG data acquired during a first time interval that the body part is in a first position that is at approximately a level of a heart of the user, and second PPG data acquired during a second time interval that the body part is in a second position that is above or below the level of the heart of the user, wherein the first PPG data and the second PPG data are associated with a first PPG morphology and a second PPG morphology, respectively, determine, as part of the calibration procedure and based on the reference PPG data, a first blood pressure metric and a second blood pressure metric associated with the first position and the second position, respectively, determine, as part of the calibration procedure, a calibration function associated with blood pressure metrics determined according to PPG data collected at a plurality of positions based at least in part on a comparison between the first blood pressure metric and the second blood pressure metric, acquire additional PPG data from the body part of the user using the wearable device during an additional time interval, the additional PPG data associated with an additional PPG morphology, determine a position of the body part of the user during the additional time interval based at least in part on a comparison between the additional PPG morphology and the first PPG morphology, the second PPG morphology, or both, determine a blood pressure metric for the user during the additional time interval based at least in part on the additional PPG data, the determined position of the body part, and the calibration function, and transmit an instruction to a user interface associated with the wearable device, a user device, or both, the instruction configured to cause the user interface to display a notification of the blood pressure metric.

Another wearable device for calibrating blood pressure measurements performed is described. The wearable device may include means for acquiring, during a calibration procedure, reference PPG data from a body part of a user using a wearable device, the reference PPG data comprising first PPG data acquired during a first time interval that the body part is in a first position that is at approximately a level of a heart of the user, and second PPG data acquired during a second time interval that the body part is in a second position that is above or below the level of the heart of the user, wherein the first PPG data and the second PPG data are associated with a first PPG morphology and a second PPG morphology, respectively, means for determining, as part of the calibration procedure and based on the reference PPG data, a first blood pressure metric and a second blood pressure metric associated with the first position and the second position, respectively, means for determining, as part of the calibration procedure, a calibration function associated with blood pressure metrics determined according to PPG data collected at a plurality of positions based at least in part on a comparison between the first blood pressure metric and the second blood pressure metric, means for acquiring additional PPG data from the body part of the user using the wearable device during an additional time interval, the additional PPG data associated with an additional PPG morphology, means for determining a position of the body part of the user during the additional time interval based at least in part on a comparison between the additional PPG morphology and the first PPG morphology, the second PPG morphology, or both, means for determining a blood pressure metric for the user during the additional time interval based at least in part on the additional PPG data, the determined position of the body part, and the calibration function, and means for transmitting an instruction to a user interface associated with the wearable device, a user device, or both, the instruction configured to cause the user interface to display a notification of the blood pressure metric.

A non-transitory computer-readable medium storing code for calibrating blood pressure measurements performed is described. The code may include instructions executable by one or more processors to acquire, during a calibration procedure, reference PPG data from a body part of a user using a wearable device, the reference PPG data comprising first PPG data acquired during a first time interval that the body part is in a first position that is at approximately a level of a heart of the user, and second PPG data acquired during a second time interval that the body part is in a second position that is above or below the level of the heart of the user, wherein the first PPG data and the second PPG data are associated with a first PPG morphology and a second PPG morphology, respectively, determine, as part of the calibration procedure and based on the reference PPG data, a first blood pressure metric and a second blood pressure metric associated with the first position and the second position, respectively, determine, as part of the calibration procedure, a calibration function associated with blood pressure metrics determined according to PPG data collected at a plurality of positions based at least in part on a comparison between the first blood pressure metric and the second blood pressure metric, acquire additional PPG data from the body part of the user using the wearable device during an additional time interval, the additional PPG data associated with an additional PPG morphology, determine a position of the body part of the user during the additional time interval based at least in part on a comparison between the additional PPG morphology and the first PPG morphology, the second PPG morphology, or both, determine a blood pressure metric for the user during the additional time interval based at least in part on the additional PPG data, the determined position of the body part, and the calibration function, and transmit an instruction to a user interface associated with the wearable device, a user device, or both, the instruction configured to cause the user interface to display a notification of the blood pressure metric.

Some examples of the method, wearable devices, and non-transitory computer-readable medium described herein may further include operations, features, means, or instructions for adjusting the second blood pressure metric by an amount to match to the reference blood pressure metric, wherein determining the calibration function may be based at least in part on the amount that the second blood pressure metric may be adjusted by to match the reference blood pressure metric.

In some examples of the method, wearable devices, and non-transitory computer-readable medium described herein, the first blood pressure metric associated with the first position that may be at approximately the level of the heart of the user may be used as a reference blood pressure metric and the calibration function comprises an algorithm or mathematical operation used to adjust blood pressure metrics collected at positions other than the first position relative to the reference blood pressure metric.

In some examples of the method, wearable devices, and non-transitory computer-readable medium described herein, acquiring the reference PPG data during the calibration procedure may include operations, features, means, or instructions for acquiring third PPG data that may be acquired from the body part of the user during a third time interval that the body part may be in a third position that may be below the level of the heart of the user, wherein the third PPG data may be associated with a third PPG morphology, and wherein the blood pressure metric may be determined based at least in part on a comparison between the additional PPG morphology and the third PPG morphology.

In some examples of the method, wearable devices, and non-transitory computer-readable medium described herein, and the method, apparatuses, and non-transitory computer-readable medium may include further operations, features, means, or instructions for receiving a user input indicating a length of the arm of the user, a height of the user, wherein determining the calibration function, determining the blood pressure metric, or both, may be based at least in part on the length of the arm of the user, an estimation of the length of the arm of the user performed based on the height of the user, or both.

In some examples of the method, wearable devices, and non-transitory computer-readable medium described herein, determining the blood pressure metric for the additional time interval may include operations, features, means, or instructions for determining an initial blood pressure metric based at least in part on the additional PPG data collected during the additional time interval and adjusting the initial blood pressure metric to determine the blood pressure metric based at least in part on the determined position of the body part and the calibration function.

In some examples of the method, wearable devices, and non-transitory computer-readable medium described herein, determining the position of the body part during the additional time interval may include operations, features, means, or instructions for inputting the first PPG data and the second PPG data into a machine learning model, extracting, using the machine learning model, a first set of reference PPG features and a second set of reference PPG features from the first PPG data and the second PPG data, respectively, inputting the additional PPG data into a machine learning classifier, extracting, using the machine learning model, an additional set of PPG features from the additional PPG data, and determining the position of the body part during the additional time interval based at least in part on a comparison between the additional set of PPG features and the first and second sets of reference PPG features.

In some examples of the method, wearable devices, and non-transitory computer-readable medium described herein, the position of the body part may be determined based at least in part on a comparison between respective sets of PPG features associated with the first PPG morphology, the second PPG morphology, and the additional PPG morphology and the respective sets of PPG features comprise a relative timing between systolic peaks and diastolic peaks, an amplitude of systolic peaks, an amplitude of diastolic peaks, a shape of a first derivative of the first PPG data, the second PPG data, the additional PPG data, or any combination thereof, a shape of a second derivative of the first PPG data, the second PPG data, the additional PPG data, or any combination thereof, a timing between peaks of the first derivative or the second derivative, or any combination thereof.

Some examples of the method, wearable devices, and non-transitory computer-readable medium described herein may further include operations, features, means, or instructions for transmitting one or more signals to the wearable device, the user device, or both, wherein the one or more signals may be configured to cause the user interface to display a set of instructions for performing the calibration procedure, wherein the set of instructions displayed by the user interface comprise a first instruction for the user to maintain the body part in the first position for the first time interval, and a second instruction for the user to maintain the body part in the second position for the second time interval.

Some examples of the method, wearable devices, and non-transitory computer-readable medium described herein may further include operations, features, means, or instructions for acquiring motion data from one or more motion sensors of the wearable device based at least in part on transmitting the one or more signals to the wearable device and confirming that the user manipulated the body part in accordance with the set of instructions displayed by the user interface based at least in part on the motion data, wherein acquiring the reference PPG data, determining the calibration function, or both, may be based at least in part on confirming that the user manipulated the body part in accordance with the set of instructions.

Some examples of the method, wearable devices, and non-transitory computer-readable medium described herein may further include operations, features, means, or instructions for receiving, via the user interface, an indication of a relative position of the body part of the user during the additional time interval, wherein the position may be determined based at least in part on the indication of the relative position.

Some examples of the method, wearable devices, and non-transitory computer-readable medium described herein may further include operations, features, means, or instructions for determining, as part of the calibration procedure and based at least in part on the reference PPG data, a first pulse transmit time associated with a first heartbeat observed at the body part in the first position, and a second pulse transmit time associated with a second heartbeat observed at the body part in the second position, wherein the first blood pressure metric and the second blood pressure metric may be based at least in part on the first pulse transmit time and the second pulse transmit time, respectively and determining, based at least in part on the additional PPG data, an additional pulse transmit time associated with an additional heartbeat observed at the body part during the additional time interval, wherein the position of the body part of the user during the additional time interval may be further based on a comparison of the additional pulse transmit time with the first pulse transmit time, the second pulse transmit time, or both.

In some examples of the method, wearable devices, and non-transitory computer-readable medium described herein, each of the first PPG data, the second PPG data, and the additional PPG data may be associated with a plurality of wavelengths and the first PPG morphology, the second PPG morphology, and the additional PPG morphology may be associated with PPG features of the plurality of wavelengths.

In some examples of the method, wearable devices, and non-transitory computer-readable medium described herein, the wearable device comprises a wearable ring device, the body part comprises a finger of the user, the wearable device comprises a wrist-worn wearable device, and the body part comprises a wrist of the user.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A method for calibrating blood pressure measurements performed by a wearable device, comprising:
acquiring, during a calibration procedure, reference photoplethysmography (PPG) data from a body part of a user using a wearable device, the reference PPG data comprising first PPG data acquired during a first time interval that the body part is in a first position that is at approximately a level of a heart of the user, and second PPG data acquired during a second time interval that the body part is in a second position that is above or below the level of the heart of the user, wherein the first PPG data and the second PPG data are associated with a first PPG morphology and a second PPG morphology, respectively;
determining, as part of the calibration procedure and based on the reference PPG data, a first blood pressure metric and a second blood pressure metric associated with the first position and the second position, respectively;
determining, as part of the calibration procedure, a calibration function associated with blood pressure metrics determined according to PPG data collected at a plurality of positions based at least in part on a comparison between the first blood pressure metric and the second blood pressure metric;
acquiring additional PPG data from the body part of the user using the wearable device during an additional time interval, the additional PPG data associated with an additional PPG morphology;
determining a position of the body part of the user during the additional time interval based at least in part on a comparison between the additional PPG morphology and the first PPG morphology, the second PPG morphology, or both;
determining a blood pressure metric for the user during the additional time interval based at least in part on the additional PPG data, the determined position of the body part, and the calibration function; and
transmitting an instruction to a user interface associated with the wearable device, a user device, or both, the instruction configured to cause the user interface to display a notification of the blood pressure metric.

2. The method of claim 1, wherein the first blood pressure metric associated with the first position that is at approximately the level of the heart of the user is used as a reference blood pressure metric, the method further comprising:
adjusting the second blood pressure metric by an amount to match to the reference blood pressure metric, wherein determining the calibration function is based at least in part on the amount that the second blood pressure metric is adjusted by to match the reference blood pressure metric.

3. The method of claim 1, wherein the first blood pressure metric associated with the first position that is at approximately the level of the heart of the user is used as a reference blood pressure metric, and wherein the calibration function comprises an algorithm or mathematical operation used to adjust blood pressure metrics collected at positions other than the first position relative to the reference blood pressure metric.

4. The method of claim 1, wherein the second PPG data is collected during the second time interval that the body part is in the second position that is above the level of the heart of the user, wherein acquiring the reference PPG data during the calibration procedure further comprises:
acquiring third PPG data that is acquired from the body part of the user during a third time interval that the body part is in a third position that is below the level of the heart of the user, wherein the third PPG data is associated with a third PPG morphology, and wherein the blood pressure metric is determined based at least in part on a comparison between the additional PPG morphology and the third PPG morphology.

5. The method of claim 1, wherein the body part comprises a hand or a finger of an arm of the user, the method further comprising:
receiving a user input indicating a length of the arm of the user, a height of the user, or both, wherein determining the calibration function, determining the blood pressure metric, or both, is based at least in part on the length of the arm of the user, an estimation of the length of the arm of the user performed based on the height of the user, or both.

6. The method of claim 1, wherein determining the blood pressure metric for the additional time interval comprises:
determining an initial blood pressure metric based at least in part on the additional PPG data collected during the additional time interval; and
adjusting the initial blood pressure metric to determine the blood pressure metric based at least in part on the determined position of the body part and the calibration function.

7. The method of claim 1, wherein determining the position of the body part during the additional time interval comprises:
inputting the first PPG data and the second PPG data into a machine learning model;
extracting, using the machine learning model, a first set of reference PPG features and a second set of reference PPG features from the first PPG data and the second PPG data, respectively;
inputting the additional PPG data into a machine learning classifier;
extracting, using the machine learning model, an additional set of PPG features from the additional PPG data; and
determining the position of the body part during the additional time interval based at least in part on a comparison between the additional set of PPG features and the first and second sets of reference PPG features.

8. The method of claim 1, wherein the position of the body part is determined based at least in part on a comparison between respective sets of PPG features associated with the first PPG morphology, the second PPG morphology, and the additional PPG morphology, and wherein the respective sets of PPG features comprise a relative timing between systolic peaks and diastolic peaks, an amplitude of systolic peaks, an amplitude of diastolic peaks, a shape of a first derivative of the first PPG data, the second PPG data, the additional PPG data, or any combination thereof, a shape of a second derivative of the first PPG data, the second PPG data, the additional PPG data, or any combination thereof, a timing between peaks of the first derivative or the second derivative, or any combination thereof.

9. The method of claim 1, further comprising:
transmitting one or more signals to the wearable device, the user device, or both, wherein the one or more signals are configured to cause the user interface to display a set of instructions for performing the calibration procedure, wherein the set of instructions displayed by the user interface comprise a first instruction for the user to maintain the body part in the first position for the first time interval, and a second instruction for the user to maintain the body part in the second position for the second time interval.

10. The method of claim 9, further comprising:
acquiring motion data from one or more motion sensors of the wearable device based at least in part on transmitting the one or more signals to the wearable device; and
confirming that the user manipulated the body part in accordance with the set of instructions displayed by the user interface based at least in part on the motion data, wherein acquiring the reference PPG data, determining the calibration function, or both, is based at least in part on confirming that the user manipulated the body part in accordance with the set of instructions.

11. The method of claim 1, further comprising:
receiving, via the user interface, an indication of a relative position of the body part of the user during the additional time interval, wherein the position is determined based at least in part on the indication of the relative position.

12. The method of claim 1, further comprising:
determining, as part of the calibration procedure and based at least in part on the reference PPG data, a first pulse transmit time associated with a first heartbeat observed at the body part in the first position, and a second pulse transmit time associated with a second heartbeat observed at the body part in the second position, wherein the first blood pressure metric and the second blood pressure metric are based at least in part on the first pulse transmit time and the second pulse transmit time, respectively; and
determining, based at least in part on the additional PPG data, an additional pulse transmit time associated with an additional heartbeat observed at the body part during the additional time interval, wherein the position of the body part of the user during the additional time interval is further based on a comparison of the additional pulse transmit time with the first pulse transmit time, the second pulse transmit time, or both.

13. The method of claim 1, wherein each of the first PPG data, the second PPG data, and the additional PPG data are associated with a plurality of wavelengths, and wherein the first PPG morphology, the second PPG morphology, and the additional PPG morphology are associated with PPG features of the plurality of wavelengths.

14. The method of claim 1,
wherein the wearable device comprises a wearable ring device, and wherein the body part comprises a finger of the user, or
wherein the wearable device comprises a wrist-worn wearable device, and wherein the body part comprises a wrist of the user.

15. A wearable device for calibrating blood pressure measurements performed, comprising:
one or more memories storing processor-executable code; and
one or more processors coupled with the one or more memories and individually or collectively operable to execute the code to cause the wearable device to:
acquire, during a calibration procedure, reference photoplethysmography (PPG) data from a body part of a user, the reference PPG data comprising first PPG data acquired during a first time interval that the body part is in a first position that is at approximately a level of a heart of the user, and second PPG data acquired during a second time interval that the body part is in a second position that is above or below the level of the heart of the user, wherein the first PPG data and the second PPG data are associated with a first PPG morphology and a second PPG morphology, respectively;
determine, as part of the calibration procedure and based on the reference PPG data, a first blood pressure metric and a second blood pressure metric associated with the first position and the second position, respectively;
determine, as part of the calibration procedure, a calibration function associated with blood pressure metrics determined according to PPG data collected at a plurality of positions based at least in part on a comparison between the first blood pressure metric and the second blood pressure metric;
acquire additional PPG data from the body part of the user during an additional time interval, the additional PPG data associated with an additional PPG morphology;
determine a position of the body part of the user during the additional time interval based at least in part on a comparison between the additional PPG morphology and the first PPG morphology, the second PPG morphology, or both;
determine a blood pressure metric for the user during the additional time interval based at least in part on the additional PPG data, the determined position of the body part, and the calibration function; and
transmit an instruction to a user interface associated with the wearable device, a user device, or both, the instruction configured to cause the user interface to display a notification of the blood pressure metric.

16. The wearable device of claim 15, wherein the one or more processors are individually or collectively further operable to execute the code to cause the wearable device to:
adjust the second blood pressure metric by an amount to match to the reference blood pressure metric, wherein determining the calibration function is based at least in part on the amount that the second blood pressure metric is adjusted by to match the reference blood pressure metric.

17. The wearable device of claim 15, wherein:
the first blood pressure metric associated with the first position that is at approximately the level of the heart of the user is used as a reference blood pressure metric, and
the calibration function comprises an algorithm or mathematical operation used to adjust blood pressure metrics collected at positions other than the first position relative to the reference blood pressure metric.

18. The wearable device of claim 15, wherein, to acquire the reference PPG data during the calibration procedure, the one or more processors are individually or collectively further operable to execute the code to cause the wearable device to:
acquire third PPG data that is acquired from the body part of the user during a third time interval that the body part is in a third position that is below the level of the heart of the user, wherein the third PPG data is associated with a third PPG morphology, and wherein the blood pressure metric is determined based at least in part on a comparison between the additional PPG morphology and the third PPG morphology.

19. The wearable device of claim 15, wherein, the body part of the user comprises a hand or a finger of an arm of the user, and the one or more processors are individually or collectively operable to execute the code to cause the wearable device to:
receive a user input indicating a length of the arm of the user, a height of the user, or both, wherein determining the calibration function, determining the blood pressure metric, or both, is based at least in part on the length of the arm of the user, an estimation of the length of the arm of the user performed based on the height of the user, or both.

20. The wearable device of claim 15, wherein, to determine the blood pressure metric for the additional time interval, the one or more processors are individually or collectively operable to execute the code to cause the wearable device to:
determine an initial blood pressure metric based at least in part on the additional PPG data collected during the additional time interval; and
adjust the initial blood pressure metric to determine the blood pressure metric based at least in part on the determined position of the body part and the calibration function.
